SEQUENCE LISTING (12 sequences)

SEQ ID NO: 1 - protein, 350 aa, Artificial Sequence
  Fetuin A isoform 1
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPADSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 2 - protein, 349 aa, Artificial Sequence
  Fetuin A isoform 2
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 3 - protein, 348 aa, Artificial Sequence
  Fetuin A isoform 3
APHGPGLIYRQPNCDDPETEEAALVAIDYINQNLPWGYKHTLNQIDEVKVWPQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 4 - protein, 321 aa, Artificial Sequence
  Fetuin A isoform 4
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 5 - protein, 283 aa, Artificial Sequence
  Fetuin A isoform Chain A
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPADSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVL

SEQ ID NO: 6 - protein, 40 aa, Artificial Sequence
  Fetuin A isoform linker
LAAPPGHQLHRAHYDLRHTFMGVVSLGSPSGEVSHPRKTR

SEQ ID NO: 7 - protein, 27 aa, Artificial Sequence
  Fetuin A isoform Chain B
TVVQPSVGAAAGPVVPPCPGRIRHFKV

SEQ ID NO: 8 - protein, 282 aa, Artificial Sequence
  Fetuin A isoform Chain A
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVL

SEQ ID NO: 9 - protein, 281 aa, Artificial Sequence
  Fetuin A isoform Chain A
APHGPGLIYRQPNCDDPETEEAALVAIDYINQNLPWGYKHTLNQIDEVKVWPQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVL

SEQ ID NO: 10 - protein, 254 aa, Artificial Sequence
  Fetuin A isoform Chain A
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKPVSSQPQPEGANEAVPTPVVDPDAPPSPPLGAPGLPPAGSPPDSHVL

SEQ ID NO: 11 - protein, 367 aa, Artificial Sequence
  FETUA_HUMAN Alpha-2-HS-glycoprotein
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCTVFQTQPVTSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 12 - protein, 19 aa, Artificial Sequence
  TGF-beta binding domain
CDFQLLKLDGKFSVVYAKC

(19) United States
(12) Patent Application Publication
(10) Pub. No.: US 2024/0101621 A1
(43) Pub. Date: Mar. 28, 2024

(54) MOLECULAR SUPERSTRUCTURE AND METHODS OF USE THEREOF

(71) Applicant: Immunis, Inc., Laguna Beach, CA (US)

(72) Inventor: Jessie Silva PAINTER, Laguna Beach, CA (US)

(21) Appl. No.: 18/274,195

(22) PCT Filed: Jan. 25, 2022
(86) PCT No.: PCT/US2022/013706

§ 371 Date: Jul. 25, 2023

Related U.S. Application Data
(60) Provisional application No. 63/191,839, filed on May 21, 2021.
(60) Provisional application No. 63/141,977, filed on Jan. 26, 2021.

(51) Int. Cl. C07K 14/47; A61K 9/00; A61K 47/61; C07K 14/495

(52) U.S. Cl. CPC C07K 14/47; A61K 9/0019; A61K 47/61; C07K 14/495

(57) ABSTRACT

Provided herein bioconjugates comprising a fetuin A-based molecule (AHSG) covalently bonded to a glycosaminoglycan (GAG) molecule.

### CROSS-REFERENCE TO RELATED APPLICATIONS

This application claims the benefit of priority under 35 U.S.C. § 119(e) from U.S. Provisional Application Ser. No. 63/141,977, filed Jan. 26, 2021, and 63/191,839, filed May 21, 2021, both of which are hereby expressly incorporated by reference in their entireties for all purposes.

### FIELD

The subject matter described herein relates generally to a molecular superstructure that includes growth factors, such as follistatin, bound to a sulphated GAG backbone.

### BACKGROUND

Aging affects all organs and tissue in the body. For example, the immune system loses the ability to protect against infections and cancer and also fails to support appropriate wound healing. At the same time, inflammatory responses mediated by the innate immune system are amplified, rendering older individuals susceptible to auto-immunity and inflammatory disease, defined as “inflammaging.” The most relevant changes are observed in the adaptive immune system, characterized by a decrease of naïve T cells and a concomitant increase in memory cells, a progressive reduction of the TCR repertoire and decreased proliferation in vitro.

The loss of muscle mass and its regeneration capacity during aging (sarcopenia), reduces strength and exercise capacity needed to perform daily living activities. Sarcopenia is evidenced by changes in innervation, stem cell function (satellite cells) and endocrine regulation of muscle homeostasis that contribute to loss of muscle fibers and decrease of functionality. After the age of 30 years, about 0.5-1% of muscle mass is lost per year in humans, with a dramatic acceleration of the rate of decline after the age of 65 years.

Dysfunctional interactions of satellite cells with muscle connective tissue fibroblasts and infiltrating macrophages and neutrophils, which normally promote muscle repair after injury, presumably contribute to decreased muscle regeneration in aged organisms. Thus, a regenerative approach to address muscle decline in aging should target both the immune system and muscle stem cell compartments.

Regenerative medicine is based on the recapitulation of normal ontogenesis, and tissue development. It employs the delivery of specific populations of live cells as a replacement therapy, or the delivery of the chemical clues that support and influence in-situ morphogenesis. Elucidation of physiological pathways and production of recombinant morphogens generically named “growth factors” have generated much interest and numerous clinical trials. The results of many of these trials have been disappointing hampered by lack of effectiveness and elevated cost of goods. Popular growth factors historically used in tissue regeneration include angiopoietin (Ang); basic fibroblast growth factor (bFGF); bone morphogenetic protein (BMP); epidermal growth factor (EGF); fibroblast growth factor (FGF); hepatocyte growth factor (HGF); insulin-like growth factor (IGF); nerve growth factor (NGF); platelet-derived growth factor (PDGF); transforming growth factor (TFG); vascular endothelial growth factor (VEGF).

The simple delivery of infusions of factors lacks targeting of specific cell populations and can result in a transient, weak biological response. The effects of supraphysiological concentrations of growth factors are hard to anticipate and, shortly after administration, due to rapid degradation or non-specific binding to tissue components, the infusion will not provide sufficient local concentration to produce the desired effect.

To address this problem, two strategies have been pursued: chemical immobilization of the growth factor into or onto a matrix and physical encapsulation of growth factors.

From a chemical point of view, non-covalent incorporation is based on absorption of growth factors by electrostatic charges. Proteins such as heparin, fibronectin, gelatin, and small oligopeptides can be chemically or physically coated to provide specific biological sites to immobilize the growth factors or morphogens. Biopolymeric gels containing fibronectin, laminin, collagen, elastin or the glycosaminoglycans heparin sulphate, chondroitin sulphate, hyaluronic acid or a variety of synthetic hydrogels can be used as extracellular matrix-mimicking materials to immobilize growth factors.

Covalent incorporation of growth factors can also provide more prolonged release. Factors may be conjugated to the polymers via functional groups, which may be incorporated by copolymerization or chemical or physical treatment. For example, epidermal growth factor (EGF) was covalently coupled to amino-silane glass via star poly(ethyleneoxide) (PEO) and TGF-β1 was conjugated covalently to poly(ethylene glycol) PEG hydrogels. The limitations of this method, however, include the unpredictability of the coupling site and compromising the growth factor bioactivity during immobilization due to damage to bioactive functional groups.

Naturally occurring materials such as silk, keratin, collagen, gelatin, fibrinogen, elastin, chitosan, hyaluronic acid, starch, carrageenan, cellulose, and alginate have also gained wide attention as drug carriers. They are often soluble in water, allowing mild fabrication conditions that are relatively harmless to the bioactivity of the growth factors.

A multiplicity of synthetic polymers, including poly(α-hydroxy acids), poly(orthoesters), poly(anhydrides), poly(amino acids), dextrin, poly(glycoside) (PGA), poly(l-lactide) (PLA) and their copolymers (PLG acid), elastin like polypeptides (ELPs), poly(ethylene glycol) (PEG) have been used for growth-factor encapsulation.

The rate of factor release from carriers can be tuned by controlling either factor diffusion or matrix degradation. A potential problem in the use of natural polymers as delivery vehicles, however, is that the degradation rate can be challenging to control, while the synthetic backbones fails to provide protection for the proteolytic degradation of the bound growth factors. Even more, fragments of the carrier polymers can cause toxicity. The bound growth factors are susceptible to enzymatic disintegration by cysteine proteases. Additionally, it was found that carrier fragments released by hydrolases generated a type II immune response with specific circulating antibodies identified in the subjects. Tunable matrix degradation and subsequent diffusion-based delivery systems were appealing for growth factor/morphogen delivery. A better delivery system, so-called “release on demand,” responded to local environmental signals triggered by pH, temperature, enzymes, or drugs that trigger cleavage of an engineered substrate. These complexes, however, still had no protease protection of the adsorbed growth factors and after degradation by hydrolases, the resulting complexes were quickly dispersed, or internalized by cells and degraded without the opportunity of growth factor-receptor interaction.

Finally, growth factor-based therapies can be unbalanced; using a single peptide or a limited combination of peptides may create unexpected results due to unbalanced receptor activation or ineffective due to lack of certain cofactors or carrier proteins. Thus, a need exists to provide a delivery vehicle that allows for the targeted delivery and prolonged and/or sustained release of growth factors.

### SUMMARY

To address this problem the use of a composition obtained from in-vitro engineered specific cell population derived from stem cells, ensure that all the factors are present in balanced proportions specific to the stage of development of the originating cells.

In some embodiments, a molecular superstructure is described that includes a fetuin A molecule (AHSG) at the core, which is connected at its N-terminus of the cystatin 1 domain with a carboxylic termination of a glycosaminoglycan (GAG) molecule. The structure may further be loaded with follistatin that is electrostatically bound to the GAG extensions at its heparin binding domain (HBD) or with other growth factors that poses HBDs.

In some embodiments, methods of producing the molecular superstructure by a synthetical process using purified individual components are described. In some embodiments, the methods of producing the structure by a self-assembling method include a cell culture of partial differentiated stem cells, a liquid media that was exposed to the cells and a set of crosslinking reagents, where the cells in culture are specifically induced to produce non-phosphorylated single chain fetuin A and at least follistatin as a growth factor with HBD.

In some embodiments, the use of the herein produced molecular superstructure to address osteo-muscular atrophy and immunomodulation of a various non-specific inflammatory conditions, including age-related inflammation, are described.

### BRIEF DESCRIPTION OF THE FIGURES

The foregoing summary, as well as the following detailed description of the invention, will be better understood when read in conjunction with the appended drawing. It should be noted that the invention is not limited to the precise embodiment shown in the drawing.

FIG. 1 is an exemplary representation of a molecular superstructure.

### DETAILED DESCRIPTION

All references and publications mentioned herein are expressly incorporated by reference in their entirety for all purposes.

Unless defined otherwise, all technical and scientific terms used herein have the same meaning as commonly understood by one of ordinary skill in the art to which this disclosure belongs. It must be noted that as used herein and in the appended claims, the singular forms “a”, “an”, and “the” include plural referents unless the context clearly dictates otherwise. Thus, for example, reference to “a peptide” includes a plurality of peptides.

The term “about” when used before a numerical designation, e.g., temperature, time, amount, and concentration, including range, indicates approximations which may vary by (+) or (−) 10%, 5% or 1%.

In certain embodiments, provided herein is a synthetic bioconjugate comprising a sulphated GAG and one or more single chain, non-phosphorylated AHSG molecule or fragment of a single chain, non-phosphorylated AHSG molecule, wherein the one or more single chain, non-phosphorylated AHSG molecule or fragment of a single chain, non-phosphorylated AHSG molecule, are covalently conjugated to the sulphated GAG.

As used herein, the term “bioconjugate” refers to a synthetic conjugate that comprises a glycosaminoglycan (GAG) and one or more AHSG molecule or AHSG fragment thereof (natural or synthetic) covalently bonded thereto. The GAG portion can be made synthetically or derived from animal sources. In some embodiments, the AHSG molecule or fragment thereof can be covalently bound to the GAG via between a terminal amino group on the AHSG molecule or AHSG fragment (e.g., the N-terminus or amino acid sidechain) and a carboxyl group on the GAG, or via a carboxyl group on the AHSG molecule or AHSG fragment (e.g., the C-terminus or amino acid sidechain) and a suitable nitrogen or oxygen atom on the GAG. In some embodiments, the term bioconjugate includes peptidoglycan.

As used herein, the term “GAG” refers to a compound having a large number of monosaccharides linked glycosidically, which also may be referred to as a “glycosaminoglycan” or “glycan”. In certain embodiments, the GAG comprises 2-aminosugars linked in an alternating fashion with uronic acids, and includes polymers such as heparin, heparan sulfate, chondroitin, keratin, and dermatan. Accordingly, non-limiting examples of GAGs which can be used in the embodiments described herein include alginate, agarose, dextran, dextran sulfate, chondroitin, chondroitin sulfate (CS), dermatan, dermatan sulfate (DS), heparan sulfate, heparin (Hep), keratin, keratan sulfate, and hyaluronic acid (HA), including derivatives thereof. In certain embodiments, the molecular weight of the GAG is varied to tailor the effects of the synthetic bioconjugate (see, e.g., Radek, K. A., et al., Wound Repair Regen., 2009, 17: 118-126; and Taylor, K. R., et al., J. Biol. Chem., 2005, 280:5300-5306, which is expressly incorporated by reference in its entirety for all purposes). In one embodiment, the GAG is degraded by oxidation and alkaline elimination (see, e.g., Fransson, L. A., et al., Eur. J. Biochem., 1980, 106:59-69, which is expressly incorporated by reference in its entirety for all purposes) to afford degraded GAG having a lower molecular weight (e.g., from about 10 kDa to about 50 kDa). In some embodiments, the GAG is unmodified. In certain embodiments, the GAG is chemically modified.

In certain embodiments, the sulphated GAG is heparan sulfate (HS), heparin (HEP), chondroitin sulfate (CS), dermatan sulfate (DS), keratan sulfate (KS), or a derivative thereof. In certain embodiments, the sulphated GAG is a chemically modified GAG. As used herein, the term “chemically modified GAG” is intended to include derivatives of GAGs (or glycosaminoglycans). For example, a chemically modified GAG can include one or more chemical derivatizations, such as, but not limited to partially N-sulfated derivatives, partially 0-sulfated derivatives, and/or partially O-carboxymethylated derivatives, or a combination thereof. In certain embodiments, the GAG is non-oxidized (i.e., does not contain oxidatively cleaved saccharide rings). In certain embodiments, the sulphated GAG is a chemically sulfated GAG.

In one embodiment, the GAG is heparin, where the heparin may include heparin derivatives, such as, but not limited to sulfated, partially N- and/or partially 0-desulfated heparin derivatives, partially O-carboxymethylated heparin derivatives, or a combination thereof. In certain embodiments, the heparin is non-oxidized heparin (i.e., does not contain oxidatively cleaved saccharide rings) and does not contain aldehyde functional groups. Heparin derivatives and/or methods for providing heparin derivatives, such as partially N-desulfated heparin and/or partially 0-desulfated heparin (i.e., 2-0 and/or 6-O-desulfated heparin) are known in the art (see, e.g., Kariya et al., J. Biol. Chem., 2000, 275:25949-5958; Lapierre, et al. Glycobiology, 1996, 6(3):355-366, which is expressly incorporated by reference in its entirety for all purposes). It is also contemplated that partially O-carboxymethylated heparin (or any GAG) derivatives, such as those which could be prepared according to Prestwich, et al. (US 2012/0142907; US 2010/0330143, which is expressly incorporated by reference in its entirety for all purposes), can be used to provide the bioconjugates disclosed herein.

In one embodiment, the GAG is dermatan sulfate (DS). The biological functions of DS are extensive, and include the binding and activation of growth factors FGF-2, FGF-7, and FGF-10, which promote endothelial cell and keratinocyte proliferation and migration. In some embodiments, the weight range of the dermatan sulfate is from about 10 kDa to about 70 kDa. In one embodiment, the molecular weight of the dermatan sulfate is about 46 kDa. In another embodiment, the dermatan sulfate is degraded by oxidation and alkaline elimination (see e.g., Fransson, L. A., et al., Eur. J. Biochem., 1980, 106:59-69, which is expressly incorporated by reference in its entirety for all purposes) to afford degraded dermatan sulfate having a low molecular weight (e.g., about 10 kDa).

Various molecular weights for the GAG can be used in the synthetic bioconjugates described herein, such as from a single disaccharide unit of about 650-700 Da to about 50 kDa. In some embodiments, the heparin is from about 10 to about 20 kDa. In some embodiments, the heparin is up to about 15, or about 16, or about 17, or about 18, or about 19, or about 20 kDa. In certain embodiments, the heparin may be oxidized under conditions that do not cleave one or more of the saccharide rings (see, e.g., Wang, et al. Biomacromolecules 2013, 14(7):2427-2432, which is expressly incorporated by reference in its entirety for all purposes).

In certain embodiments, the synthetic bioconjugate comprises one or more single chain, non-phosphorylated AHSG molecule or fragment of a single chain, non-phosphorylated AHSG molecule are covalently conjugated to the sulphated GAG at the N-terminus. In certain embodiments, the AHSG molecule or fragment thereof is covalently conjugated to the sulphated GAG at the N-terminus via an amide bond.

In certain embodiments, the AHSG molecule or AHSG fragment is bonded to the sulphated GAG via a linker. As used herein, the term “linker” is intended to refer to an optional portion of the bioconjugate which links the AHSG molecule or AHSG fragment to the GAG. In such embodiments, the linker can comprise from 1-100 linear chain atoms and be cleavable or non-cleavable. In certain embodiments, the linker is non-cleavable. In certain embodiments, the linker comprises a peptide linker (e.g., 1-10 or 1-5 amino acids). It is contemplated that any amino acid, natural or unnatural, can be used in the linker sequence, provided that the linker sequence does not significantly interfere with the intended binding of the AHSG molecule or AHSG fragment. The linker can be bound to any suitable amino acid of the AHSG molecule or AHSG fragment (including a sidechain, the C-terminus or N-terminus). In certain embodiments, the AHSG molecule or AHSG fragment is covalently conjugated to the backbone of the sulphated GAG through a linker.

In various embodiments described herein, the AHSG molecule or AHSG fragment can be modified by the inclusion of one or more conservative amino acid substitutions. As is well known to those skilled in the art, altering any non-critical amino acid of a peptide by conservative substitution should not significantly alter the activity of that peptide because the side-chain of the replacement amino acid should be able to form similar bonds and contacts to the side chain of the amino acid which has been replaced. Non-conservative substitutions may too be possible, provided that they do not substantially affect the binding activity of the AHSG molecule or AHSG fragment.

In certain embodiments, the one or more single chain, non-phosphorylated AHSG molecules or fragment of a single chain, non-phosphorylated AHSG molecule has at least about 80% sequence identity to fetuin-A. As used herein, the term “sequence identity” refers to a level of amino acid residue or nucleotide identity between two peptides or proteins. When a position in the compared sequence is occupied by the same amino acid, then the molecules are identical at that position. A peptide (or a polypeptide or peptide region) has a certain percentage (for example, at least about 60%, or at least about 65%, or at least about 70%, or at least about 75%, or at least about 80%, or at least about 83%, or at least about 85%, or at least about 90%, or at least about 95%, or at least about 98% or at least about 99%) of “sequence identity” to another sequence means that, when aligned, that percentage of amino acids are the same in comparing the two sequences. It is noted that, for any sequence (“reference sequence”) disclosed herein, sequences having at least about 60%, or at least about 65%, or at least about 70%, or at least about 75%, or at least about 80%, or at least about 83%, or at least about 85%, or at least about 90%, or at least about 95%, or at least about 98%, or at least about 99% sequence identity to the reference sequence are also within the disclosure. Likewise, the present disclosure also includes sequences that have one, two, three, four, or five substitution, deletion or addition of amino acid residues as compared to the reference sequences. In certain embodiments, in any one or more of the sequences specified herein, the sequence may be modified by having one, two, three, or up to five, or up to ten, or up to twenty amino addition, deletion and/or substitution each therefrom.

In certain embodiments, the fragment of a single chain, non-phosphorylated AHSG molecule comprises a TGFβ-binding sequence of fetuin-A. In certain embodiments, the fragment of a single chain, non-phosphorylated AHSG molecule has at least 80% or at least about 83%, or at least about 85%, or at least about 90%, or at least about 95%, or at least about 96%, or at least about 97%, or at least about 98%, or at least about 99% sequence identity, to the TGFβ-binding sequence of fetuin-A.

In certain embodiments, the fragment of a single chain, non-phosphorylated AHSG molecule comprises an amino acid sequence selected from the group consisting of SEQ ID NO:1-4 or SEQ ID NO:11. In certain embodiments, the fragment of a single chain, non-phosphorylated AHSG molecule comprises SEQ ID NO:1. In certain embodiments, the fragment of a single chain, non-phosphorylated AHSG molecule comprises SEQ ID NO:2. In certain embodiments, the fragment of a single chain, non-phosphorylated AHSG molecule comprises SEQ ID NO:3. In certain embodiments, the fragment of a single chain, non-phosphorylated AHSG molecule comprises SEQ ID NO:4. In certain embodiments, the fragment of a single chain, non-phosphorylated AHSG molecule comprises SEQ ID NO:11.

In certain embodiments, the fragment of a single chain, non-phosphorylated AHSG molecule comprises an amino acid sequence selected from the group consisting of SEQ ID NO:1-4 or SEQ ID NO:11, or is a fragment of any one of SEQ ID NO:1-4 or SEQ ID NO:11 that comprises the fragment of SEQ ID NO:12. In certain embodiments, the fragment of a single chain, non-phosphorylated AHSG molecule comprises SEQ ID NO:1, or a fragment of SEQ ID NO:1 that comprises the fragment of SEQ ID NO:12. In certain embodiments, the fragment of a single chain, non-phosphorylated AHSG molecule comprises SEQ ID NO:2, or a fragment of SEQ ID NO:2 that comprises the fragment of SEQ ID NO:12. In certain embodiments, the fragment of a single chain, non-phosphorylated AHSG molecule comprises SEQ ID NO:3, or a fragment of SEQ ID NO:3 that comprises the fragment of SEQ ID NO:12. In certain embodiments, the fragment of a single chain, non-phosphorylated AHSG molecule comprises SEQ ID NO:4, or a fragment of SEQ ID NO:4 that comprises the fragment of SEQ ID NO:12. In certain embodiments, the fragment of a single chain, non-phosphorylated AHSG molecule comprises SEQ ID NO:11, or a fragment of SEQ ID NO:11 that comprises the fragment of SEQ ID NO:12.

In certain embodiments, the fragment of a single chain, non-phosphorylated AHSG molecule comprises the amino acid sequence of SEQ ID NO:12 or an amino acid sequence having at least 85% sequence identity to SEQ ID NO:12. In certain embodiments, the fragment of a single chain, non-phosphorylated AHSG molecule comprises has at least 80% sequence identity, at least 90% sequence identity, at least 95% sequence identity, or at least 99% sequence identity, to SEQ ID NO:12.

In certain embodiments, the fragment of a single chain, non-phosphorylated AHSG molecule comprises from about 3 to about 120 amino acids, or from about 3 to about 110 amino acids, or from about 3 to about 100 amino acids, or from about 3 to about 90 amino acids, or from about 3 to about 80 amino acids, or from about 3 to about 70 amino acids, or from about 3 to about 60 amino acids, or from about 3 to about 50 amino acids, or from about 3 to about 40 amino acids, or from about 5 to about 120 amino acids, or from about 5 to about 100 amino acids, or from about 5 to about 90 amino acids, or from about 5 to about 80 amino acids, or from about 5 to about 70 amino acids, or from about 5 to about 60 amino acids, or from about 5 to about 50 amino acids, or from about 5 to about 40 amino acids, or from about 5 to about 30 amino acids, or from about 5 to about 20 amino acids, or from about 5 to about 10 amino acids.

In certain embodiments, the number of single chain, non-phosphorylated AHSG molecules or fragments thereof per GAG may be described as a “percent (%) functionalization” based on the percent of disaccharide units which are functionalized with AHSG molecules or fragments thereof on the GAG backbone. For example, the total number of available disaccharide units present on the GAG can be calculated by dividing the molecular weight (or the average molecular weight) of a single disaccharide unit (e.g., about 550-800 Da, or from about 650-750 Da) by the molecular weight of the GAG (e.g., about 25 kDa up to about 70 kDa, or even about 100 kDa). In embodiments where the GAG does not contain conventional “disaccharide units” (e.g., alginic acid), the total number of available disaccharide units present on the GAG to be used in the calculations presented herein, can be calculated by dividing the molecular weight (or the average molecular weight) of a single saccharide unit by the molecular weight of the GAG, and multiplying by 2.

Therefore, in certain embodiments, the GAG comprises from about 1% to about 50%, or from about 5% to about 30% functionalization with AHSG molecules or fragments thereof, or from about 10% to about 30% functionalization with AHSG molecules or fragments thereof, or about 20% functionalization with AHSG molecules or fragments thereof, wherein the percent (%) functionalization with AHSG molecules or fragments thereof is determined by a percent of disaccharide units on the GAG which are functionalized with AHSG molecules or fragments thereof. In some embodiments, the percent (%) functionalization of the GAG with AHSG molecules or fragments thereof is from about 1% to about 75%, 1% to about 50%, or from about 3% to about 40%, or from about 5% to about 30%, or from about 10% to about 30%, or about 5%, or about 10%, or about 15%, or about 20%, or about 25%, or about 30%, or about 35%, or about 40%, or about 45%, or about 50%. In certain embodiments, the sulphated GAG comprises from about 1% to about 75 percent (%) functionalization, wherein the percent (%) functionalization is determined by a percent of disaccharide units on the sulphated GAG which are functionalized with a single chain, non-phosphorylated AHSG molecule or fragment of a single chain, non-phosphorylated AHSG molecule.

In certain embodiments, the synthetic bioconjugate comprises from 1 to about 20, or from 1 to about 15, or from 1 to about 12, or from 1 to about 10, or from 1 to about 9, or from 1 to about 8, or from 1 to about 7, or from 1 to about 6, or from 1 to 5, or from 1 to 4, or from 1 to 3, or from 1 to 2 single chain, non-phosphorylated AHSG molecules or fragment of a single chain, non-phosphorylated AHSG molecule. In certain embodiments, the synthetic bioconjugate comprises from 1 to 5 single chain, non-phosphorylated AHSG molecules or fragment of a single chain, non-phosphorylated AHSG molecule.

In certain embodiments, the number of AHSG molecules or fragments thereof varies in the composition. In any of the embodiments described herein, the number of AHSG molecules or fragments thereof per sulphated GAG is an average, where certain bioconjugates in a composition may have more AHSG molecules or fragments thereof per sulphated GAG and certain synthetic bioconjugates have less AHSG molecules or fragments thereof per sulphated GAG. Accordingly, in certain embodiments, the number of AHSG molecules or fragments thereof as described herein is an average in a composition of synthetic bioconjugates. For example, in certain embodiments, the synthetic bioconjugates are a composition where the average number of AHSG molecules or fragments thereof per GAG is about 5. In other embodiments, the average number of single chain, non-phosphorylated AHSG molecule or fragment of a single chain, non-phosphorylated AHSG molecule per sulphated GAG is from 1 to about 20, or from 1 to about 15, or from 1 to about 12, or from 1 to about 10, or from 1 to about 9, or from 1 to about 8, or from 1 to about 7, or from 1 to about 6, or from 1 to 5, or from 1 to 4, or from 1 to 3, or from 1 to 2.

In certain embodiments, the synthetic bioconjugate comprises one single chain, non-phosphorylated AHSG molecule or fragment of a single chain, non-phosphorylated AHSG molecule.

In certain embodiments, the synthetic bioconjugate comprises a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of a single chain, non-phosphorylated AHSG molecule.

In one embodiment, the synthetic bioconjugates of the disclosure bind, either directly or indirectly, to follistatin. The terms “binding” or “bind” as used herein are meant to include interactions between molecules that may be detected using, for example, a hybridization assay, surface plasmon resonance, ELISA, competitive binding assays, isothermal titration calorimetry, phage display, affinity chromatography, rheology or immunohistochemistry. The terms are also meant to include “binding” interactions between molecules. Binding may be “direct” or “indirect.” “Direct” binding comprises direct physical contact between molecules. “Indirect” binding between molecules comprises the molecules having direct physical contact with one or more molecules simultaneously. This binding can result in the formation of a “complex” comprising the interacting molecules. A “complex” refers to the binding of two or more molecules held together by covalent or non-covalent bonds, interactions or forces. Accordingly, also provided is a composition comprising the synthetic bioconjugate as disclosed herein, or a composition comprising the same, wherein the composition further comprises follistatin. In certain embodiments, the follistatin is present in a 1- to 5-fold excess of the synthetic bioconjugate. In certain embodiments, the follistatin is present in a 5-fold excess, or a 4-fold excess, or a 3-fold excess, or a 2-fold excess, or in a 1:1 ratio with the synthetic bioconjugate.

In certain embodiments, the follistatin is present in a 1:1 ratio of follistatin per disaccharide unit on the GAG. In certain embodiments, the follistatin is present in a 1:2 of follistatin per disaccharide unit on the GAG. In certain embodiments, the follistatin is present in up to a 1:2 of follistatin per disaccharide unit on the GAG. In certain embodiments, the composition of synthetic bioconjugates compsrises an average ratio of from 2:1 to 1:2, or from 1:1 to 1:2, follistatin per disaccharide unit on the GAG.

In certain embodiments, the molecular superstructure disclosed herein provides a) binding of one or more growth factors that possesses HB domain, b) protection of the growth factor against cysteine proteases, c) anti-inflammatory effect, d) TGFb sequestration and e) mineral sequestration. The superstructure has a sulphated GAG backbone that is functionalized at the carboxylic terminations with natural AHSG molecules by covalent ester linkage at the amino-terminations of the cystatin domain 1.

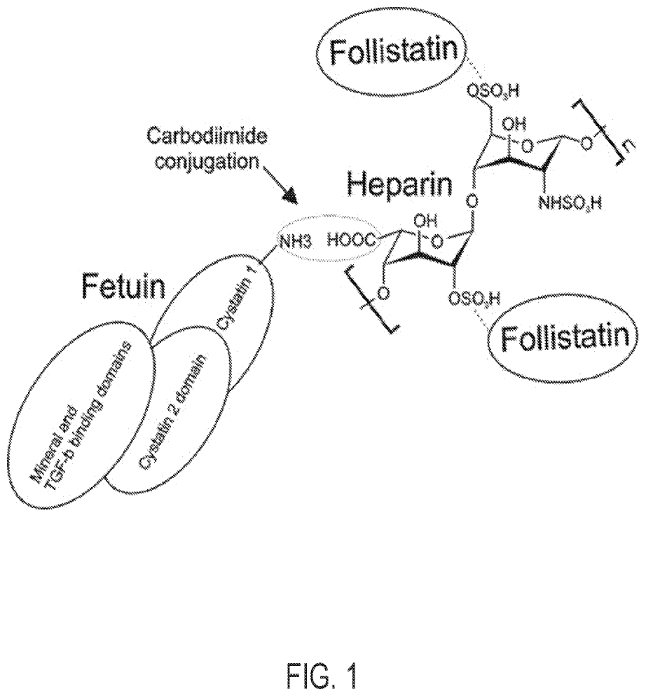

The construct further attaches growth factors. As seen in FIG. 1, an exemplary GAG is heparin, and an exemplary growth factor is follistatin, however other GAGs or growth factors may be attached. A molecular superstructure may include a fetuin A molecule (AHSG) at the core, which is connected at its N-terminus of the cystatin 1 domain with a carboxylic termination of a glycosaminoglycan (GAG) molecule. The structure may further be loaded with follistatin that is electrostatically bound to the GAG extensions at its heparin binding domain (HBD) or with other growth factors that poses HBDs.

Carbodiimide coupling chemistry presents one of the most popular approaches for covalently grafting molecules to various substrates. Among these coupling reagents, the prevalent crosslinking molecule is 1-ethyl-3-(3-dimethylamminopropyl) carbodiimide (EDC) for bioconjugation purposes. EDC mediates the conjugation reaction between carboxylic acid groups and amino groups, producing the formation of stable intermolecular amide bonds in aqueous environments at physiological pH.

Compositions

In one embodiment, the bioconjugate is administered in a composition. The present disclosure provides compositions comprising a bioconjugate and a pharmaceutically acceptable carrier for administration, such as for local or systemic administration. In certain embodiments, the composition is formulated for local administration, such as, but not limited to, intramuscular or intra-articular injection. In certain embodiments, the composition is formulated for systemic administration or injection, such as, but not limited to, intramuscular or intravenous administration. In certain embodiments, the mode of administration is dependent on the size of the bioconjugate, such as, for example, a shorter backbone may be employed for systemic administration.

Pharmaceutically acceptable carriers are known to one having ordinary skill in the art may be used, including water or saline. As is known in the art, the components as well as their relative amounts are determined by the intended use and method of delivery. Diluent or carriers employed in the compositions can be selected so that they do not diminish the desired effects of the bioconjugate. Examples of suitable compositions include aqueous solutions, for example, a solution in isotonic saline, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents.

In certain embodiments, the composition is an aqueous solution. Aqueous solutions are suitable for use in composition formulations based on ease of formulation, as well as an ability to easily administer such compositions by means of instilling the solution in. In certain embodiments, the compositions are suspensions, viscous or semi-viscous gels, or other types of solid or semi-solid compositions. In some embodiments, the composition is in the form of foams, ointments, liquid wash, gels, sprays and liposomes, which are very well known in the art. Alternatively, the topical administration is an infusion of the provided bioconjugate to the treatment site via a device selected from a pump-catheter system, a continuous or selective release device, or an adhesion barrier. In certain embodiments, the composition is a solution that is directly applied to or contacts the internal wall of a vein or artery. In some embodiments, the composition comprises a polymer matrix. In other embodiments, the composition is absorbable. In certain embodiments, the composition comprises a pH buffering agent. In some embodiments, the composition contains a lubricity enhancing agent.

In certain embodiments, a polymer matrix or polymeric material is employed as a pharmaceutically acceptable carrier or support for the composition. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, the compositions provided herein is formulated as films, gels, foams, or and other dosage forms.

Suitable ionic strength modifying agents include, for example, glycerin, propylene glycol, mannitol, glucose, dextrose, sorbitol, sodium chloride, potassium chloride, and other electrolytes.

In certain embodiments, the solubility of the bioconjugate may need to be enhanced. In such cases, the solubility may be increased by the use of appropriate formulation techniques, such as the incorporation of solubility-enhancing compositions such as mannitol, ethanol, glycerin, polyethylene glycols, propylene glycol, poloxomers, and others known in the art.

In certain embodiments, the composition contains a lubricity enhancing agent. As used herein, lubricity enhancing agents refer to one or more pharmaceutically acceptable polymeric materials capable of modifying the viscosity of the pharmaceutically acceptable carrier. Suitable polymeric materials include, but are not limited to: ionic and non-ionic water soluble polymers; hyaluronic acid and its salts, chondroitin sulfate and its salts, dextrans, gelatin, chitosans, gellans, other bioconjugates or polysaccharides, or any combination thereof; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; collagen and modified collagens; galactomannans, such as guar gum, locust bean gum and tara gum, as well as polysaccharides derived from the foregoing natural gums and similar natural or synthetic gums containing mannose and/or galactose moieties as the main structural components (e.g., hydroxypropyl guar); gums such as tragacanth and xanthan gum; gellan gums; alginate and sodium alginate; chitosans; vinyl polymers; hydrophilic polymers such as polyethylene oxides, polyoxyethylene-polyoxypropylene copolymers, and polyvinylalcohol; carboxyvinyl polymers or crosslinked acrylic acid polymers such as the “carbomer” family of polymers, e.g., carboxypolyalkylenes that may be obtained commercially under the Carbopol™ trademark; and various other viscous or viscoelastomeric substances. In one embodiment, a lubricity enhancing agent is selected from the group consisting of hyaluronic acid, dermatan, chondroitin, heparin, heparan, keratin, dextran, chitosan, alginate, agarose, gelatin, hydroxypropyl cellulose, hydroxyethyl cellulose, hydroxypropyl methylcellulose, hydroxypropyl methylcellulose phthalate, methyl cellulose, carboxymethyl cellulose, and etherified cellulose, polyvinyl alcohol, polyvinylpyrrolidinone, povidone, carbomer 941, carbomer 940, carbomer 971P, carbomer 974P, or a pharmaceutically acceptable salt thereof. In one embodiment, a lubricity enhancing agent is applied concurrently with the bioconjugate. Alternatively, in one embodiment, a lubricity enhancing agent is applied sequentially to the bioconjugate. In one embodiment, the lubricity enhancing agent is chondroitin sulfate. In one embodiment, the lubricity enhancing agent is hyaluronic acid. The lubricity enhancing agent can change the viscosity of the composition.

In some embodiments, the bioconjugates can be combined with minerals, amino acids, sugars, peptides, proteins, vitamins (such as ascorbic acid), or laminin, collagen, fibronectin, hyaluronic acid, fibrin, elastin, or aggrecan, or growth factors such as epidermal growth factor, platelet-derived growth factor, transforming growth factor beta, or fibroblast growth factor, and glucocorticoids such as dexamethasone or viscoelastic altering agents, such as ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic.

Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or IVIES. In certain embodiments, an appropriate buffer system (e.g., sodium phosphate, sodium acetate, sodium citrate, sodium borate or boric acid) is added to the composition to prevent pH drift under storage conditions. In some embodiments, the buffer is a phosphate buffered saline (PBS) solution (i.e., containing sodium phosphate, sodium chloride and in some formulations, potassium chloride and potassium phosphate). The particular concentration will vary, depending on the agent employed. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8. In some embodiments, the buffer is chosen to maintain a pH within the range of from about pH 4 to about pH 8. In some embodiments, the pH is from about pH 5 to about pH 8. In some embodiments, the buffer is a saline buffer. In certain embodiments, the pH is from about pH 4 and about pH 8, or from about pH 3 to about pH 8, or from about pH 4 to about pH 7. In some embodiments, the composition is in the form of a film, gel, patch, or liquid solution which comprises a polymeric matrix, pH buffering agent, a lubricity enhancing agent and a bioconjugate wherein the composition optionally contains a preservative; and wherein the pH of said composition is within the range of about pH 4 to about pH 8.

The bioconjugate may be sterilized to remove unwanted contaminants including, but not limited to, endotoxins and infectious agents. Sterilization techniques which do not adversely affect the structure and biotropic properties of the bioconjugate can be used. In certain embodiments, the bioconjugate can be disinfected and/or sterilized using conventional sterilization techniques including propylene oxide or ethylene oxide treatment, sterile filtration, gas plasma sterilization, gamma radiation, electron beam, and/or sterilization with a peracid, such as peracetic acid. In one embodiment, the bioconjugate can be subjected to one or more sterilization processes. Alternatively, the bioconjugate may be wrapped in any type of container including a plastic wrap or a foil wrap, and may be further sterilized.

In some embodiments, preservatives are added to the composition to prevent microbial contamination during use. Suitable preservatives added to the compositions comprise benzalkonium chloride, benzoic acid, alkyl parabens, alkyl benzoates, chlorobutanol, chlorocresol, cetyl alcohols, fatty alcohols such as hexadecyl alcohol, organometallic compounds of mercury such as acetate, phenylmercury nitrate or borate, diazolidinyl urea, diisopropyl adipate, dimethyl polysiloxane, salts of EDTA, vitamin E and its mixtures. In certain embodiments, the preservative is selected from benzalkonium chloride, chlorobutanol, benzododecinium bromide, methyl paraben, propyl paraben, phenylethyl alcohol, edentate disodium, sorbic acid, or polyquarternium-1. In certain embodiments, the compositions comprise a preservative. In some embodiments, the preservatives are employed at a level of from about 0.001% to about 1.0% w/v. In certain embodiments, the compositions do not contain a preservative and are referred to as “unpreserved”. In some embodiments, the unit dose compositions are sterile, but unpreserved.

In some embodiments, separate or sequential administration of the bioconjugate and other agent is necessary to facilitate delivery of the composition. In certain embodiments, the bioconjugate and the other agent can be administered at different dosing frequencies or intervals. For example, the bioconjugate can be administered daily, while the other agent can be administered less frequently. Additionally, as will be apparent to those skilled in the art, the bioconjugate and the other agent can be administered using the same route of administration or different routes of administration.

Any effective regimen for administering the bioconjugate can be used. For example, the bioconjugate can be administered as a single dose, or as a multiple-dose daily regimen. Further, a staggered regimen, for example, one to five days per week can be used as an alternative to daily treatment.

In various embodiments, the bioconjugate can be administered topically, such as by film, gel, patch, or liquid solution. In some of the embodiments, the compositions provided are in a buffered, sterile aqueous solution. In certain embodiments, the solutions have a viscosity of from about 1 to about 100 centipoises (cps), or from about 1 to about 200 cps, or from about 1 to about 300 cps, or from about 1 to about 400 cps. In some embodiments, the solutions have a viscosity of from about 1 to about 100 cps. In certain embodiments, the solutions have a viscosity of from about 1 to about 200 cps. In certain embodiments, the solutions have a viscosity of from about 1 to about 300 cps. In certain embodiments, the solutions have a viscosity of from about 1 to about 400 cps. In certain embodiments, the solution is in the form of an injectable liquid solution. In other embodiments, the compositions are formulated as viscous liquids, i.e., viscosities from several hundred to several thousand cps, gels or ointments. In these embodiments, the bioconjugate is dispersed or dissolved in an appropriate pharmaceutically acceptable carrier.

Exemplary compositions contemplated by the present disclosure may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Films used for drug delivery are well known in the art and comprise non-toxic, non-irritant polymers devoid of leachable impurities, such as polysaccharides (e.g., cellulose, maltodextrin, etc.). In some embodiments, the polymers are hydrophilic. In other embodiments, the polymers are hydrophobic. The film adheres to tissues to which it is applied, and is slowly absorbed into the body over a period of about a week. Polymers used in the thin-film dosage forms described herein are absorbable and exhibit sufficient peel, shear and tensile strengths as is well known in the art. In some embodiments, the film is injectable. In certain embodiments, the film is administered to the patient prior to, during or after surgical intervention.

Gels are used herein refer to a solid, jelly-like material that can have properties ranging from soft and weak to hard and tough. As is well known in the art, a gel is a non-fluid colloidal network or polymer network that is expanded throughout its whole volume by a fluid. A hydrogel is a type of gel which comprises a network of polymer chains that are hydrophilic, sometimes found as a colloidal gel in which water is the dispersion medium. Hydrogels are highly absorbent and can contain a high degree of water, such as, for example greater than 90% water. In some embodiments, the gel described herein comprises a natural or synthetic polymeric network. In some embodiments, the gel comprises a hydrophilic polymer matrix. In other embodiments, the gel comprises a hydrophobic polymer matrix. In some embodiments, the gel possesses a degree of flexibility very similar to natural tissue. In certain embodiments, the gel is biocompatible and absorbable. In certain embodiments, the gel is administered to the patient prior to, during or after surgical intervention.

Exemplary formulations may comprise: a) one or more bioconjugate as described herein; b) pharmaceutically acceptable carrier; and c) hydrophilic polymer as matrix network, wherein said compositions are formulated as viscous liquids, i.e., viscosities from several hundred to several thousand cps, gels or ointments. In these embodiments, the bioconjugate is dispersed or dissolved in an appropriate pharmaceutically acceptable carrier.

In certain embodiments, the bioconjugate, or a composition comprising the same, is lyophilized prior to, during, or after, formulation. Accordingly, also provided herein is a lyophilized composition comprising a bioconjugate or composition comprising the same as described herein.

Suitable dosages of the bioconjugate can be determined by standard methods, for example by establishing dose-response curves in laboratory animal models or in clinical trials and can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In various exemplary embodiments, a dose ranges from about 0.01 μg to about 10 g. For example, for systemic delivery, the dose can be about 10 g, or about 5 g, or about 1 g. In other illustrative embodiments, effective doses ranges from about 100 μg to about 10 g per dose, or from about 100 μg to about 1 g per dose, or from about 100 μg to about 500 mg per dose, from about 0.01 μg to about 100 mg per dose, or from about 100 μg to about 50 mg per dose, or from about 500 μg to about 10 mg per dose, or from about 1 mg to 10 mg per dose, or from about 1 to about 100 mg per dose, or from about 1 mg to 500 mg per dose, or from about 1 mg to 200 mg per dose, or from about 10 mg to 100 mg per dose, or from about 10 mg to 75 mg per dose, or from about 10 mg to 50 mg per dose, or about 10 mg per dose, or about 20 mg per dose, or about 30 mg per dose, or about 40 mg per dose, or about 50 mg per dose, or about 60 mg per dose, or about 70 mg per dose, or about 80 mg per dose, or about 90 mg per dose, or about 100 mg per dose. In any of the various embodiments described herein, effective doses ranges from about 0.01 μg to about 1000 mg per dose, 1 μg to about 100 mg per dose, about 100 μg to about 1.0 mg, about 50 μg to about 600 μg, about 50 μg to about 700 μg, about 100 μg to about 200 μg, about 100 μg to about 600 μg, about 100 μg to about 500 μg, about 200 μg to about 600 μg, or from about 100 μg to about 50 mg per dose, or from about 500 μg to about 10 mg per dose or from about 1 mg to about 10 mg per dose.

In certain embodiments, the daily dosages appropriate for the compounds described herein described herein are from about 0.01 to about 2.5 mg/kg per body weight. In some embodiments, an indicated daily dosage in the larger subject, including, but not limited to, humans, is in the range from about 0.5 mg to about 100 mg, conveniently administered in divided doses, including, but not limited to, up to four times a day or in extended-release form. In certain embodiments, suitable unit dosage forms for oral administration comprise from about 1 to about 50 mg active ingredient. The foregoing ranges are merely suggestive, as the number of variables in regard to an individual treatment regime is large, and considerable excursions from these recommended values are not uncommon. In certain embodiments, the dosages are altered depending on a number of variables, not limited to the activity of the compound used, the disease or condition to be treated, the mode of administration, the requirements of the individual subject, the severity of the disease or condition being treated, and the judgment of the practitioner.

In certain embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In certain embodiments, compounds exhibiting high therapeutic indices are preferred. In some embodiments, the data obtained from cell culture assays and animal studies is used in formulating a range of dosage for use in human. In specific embodiments, the dosage of such compounds lies within a range of circulating concentrations that include the ED₅₀ with minimal toxicity. In certain embodiments, the dosage varies within this range depending upon the dosage form employed and the route of administration utilized.

### EXAMPLES

### Example 1: General Procedure for Chemically Sulfated Glycosaminoglycan (GAG)

Glycosaminoglycans (e.g., dextran, chondroitin, dermatan, heparin, keratin, hyaluronic acid, etc.) can be purchased from commercial sources. The glycosaminoglycan is converted to a tetrabutylamine salt, dissolved in N,N-dimethylformamide to which N,N-dimethylformamide sulfur trioxide complex dissolved in N,N-dimethylformamide is added. After 1 h at 70° C., the reaction is stopped by adding cold water. The pH is adjusted with 10 N NaOH to pH 9. The sulfated material is precipitated with 3 volumes of ethanol saturated with sodium acetate and collected by centrifugation. The resulting pellet is washed with 75% ethanol three times using ultrasound to break up the pellet followed by centrifugation. The sulfated material is dissolved in water, dialyzed to remove salt, and lyophilized.

### Example 2: The Synthesis of Synthetic Bioconjugate Using Purified Components is Accomplished by the Following Steps

1. One, or a mixture of sulphated GAGs [heparan sulfate (HS) heparin (HEP), chondroitin sulfate (CS), dermatan sulfate (DS), or keratan sulfate (KS)] are activated utilizing a carbodiimide linking agent at 2-4° C. A common method uses 1-ethyl-3-(3-dimethylaminopropyl)-carbodiimide/N-hydroxysulfoxuccinimide (EDC/sNHS) to activate the carboxylic acid moieties on GAGs. Use low molecular weight GAGs for a soluble gel or higher molecular weight for a more consistent gel.
2. AHSG at the 2:1 molar ratio with the GAG monomers is added and incubated for 15 minutes at 8° C. on thermomixer. In this step, the N-terminus of the cystatin domain 1 of the AHSG is coupled to the EDC/sNHS-activated carboxylic acid groups of GAG to form defined biohybrid matrices.
3. The composition is washed by distilled water dialysis for at least 5 hours.
4. The composition is re-suspended in a physiological buffer.
5. Growth factor is added to the solution at 2:1 to molar GAG monomeric ratio.
6. Adjuvants (aminoacids, peptides, cytokines, N-acetylcysteine, etc) are added to the solution.
7. The solution volume is adjusted to final concentration with the physiological buffer.
8. The solution is gamma-irradiated and packaged in sterile containers.

An alternative synthesis uses a cell culture media composition that was previously exposed to a cell culture (conditioned media, CM) that is producing both GAGs and growth factors. For this approach, the steps include:
1. CM is collected and filtered through a 0.1 μm filter membrane.
2. The composition is washed by distilled water dialysis for at least 3 hours.
3. EDC/NHS is added at cold (8° C.) on the thermomixer and agitated at low speed for 30 minutes.
4. The composition is washed by distilled water dialysis for at least 5 hours.
5. Adjuvants (growth factors, amino acids, peptides, cytokines, N-acetylcysteine, etc.) are added to the solution.
6. The solution is gamma-irradiated and packaged in sterile containers.

### Example 3. An In-Vitro System to Test the Bioconjugates Includes an Vitro Cell Culture where the Bioconjugates are Added in Various Concentrations to Establish a Dose Effect Relation

Such system includes a culture of primary myoblasts that are propagated in vitro in conditions that does not allow differentiation. The bioconjugate is tested to evaluate the rate of growth of muscle progenitors (myoblast). The bioconjugate is further tested to evaluate on the same in-vitro system for myoblast differentiation, fusion rate, content in nuclei and mitochondria and ATP content.

The effect on the immune system is tested by cytokine release in a peripheral blood mononuclear cell culture (PBMC). Various doses of the bioconjugate are added to the PBMC culture for 24-48 hours, then the supernatant is analyzed for cytokines by ELISA or Luminex assays. The PBMCs are then analyzed for various lymphocyte by flowcytometry or by intracellular staining (ICS) for additional cytokines.

Additional studies are designed to study the effect on other cell populations, including neurons, osteocytes, chondrocytes, epithelial cells, hepatocytes, cardiomyocytes, adipocytes and other cells.

In vivo experiments are performed to assess the safety and efficacy of the bioconjugates. Young, adult, and aged animals are used in various dosing regimens to establish the effect on muscle, joints, immune system, metabolism, adipogenesis, cognitive function, by established testing methods.

One of the advantages using this method includes the inclusion of the entire secretome of the specialized cell culture, however the exact structure is less predictable as carbodiimide coupling reactions may engage the amino groups of any molecules in the mixture. To prevent coupling of small amino-acids and peptides that are present in the media, an initial dialysis with cutoff molecular weight of 10 kDa is performed on the CM.

The composition described herein provides a carrier or a slow release construct. The composition is hydrophilic, and the release of the electrostatically bound growth factors is by slow diffusion or enzymatic degradation of the backbone.

Human fetuin-A (formerly called α2HS-glycoprotein, AHSG) is a multifunctional glycoprotein which is secreted ubiquitously in young organisms, almost exclusively by the liver parenchymal cells in adulthood. The cell-culture-derived AHSG is secreted in a non-phosphorylated single-chain form as opposed to the two-chain partial phosphorylated form isolated from human serum.

Advantages using AHSG as functional molecule:
1. Broad-range protease inhibition. The two cystatin domains in the AHSG composition belongs to the cystatin superfamily of cysteine protease inhibitors, thus conferring protection to enzymatic degradation of the attached growth factors.
2. Fetuin-A is also known a negative acute phase reactant with anti-inflammatory characteristics. AHSG provides an anti-inflammatory effect by directly inhibiting PAMP-induced HMGB1 release by innate immune cells.
3. In addition, AHSG is a mineral chaperone, attaches to hydroxyapatite crystals and inhibits calcification both in vitro and in vivo. Tissue, and particularly vascular calcification, are a well-known phenomenon in aging.
4. Suppression of TGFb1 by AHSG's specific binding domain, allows the inhibition of many negative aspects of aging.

Many growth factors, such as BMP-2, BMP-7, VEGF, PDG and FGF-2, interact specifically with the sulfated GAGs of the ECM. The construct described herein is particularly focused on the use of follistatin (FST), however does not exclude the use of other growth factors or peptides with similar biological activity.

Follistatin is a multi-domain protein that includes an N-terminal domain and three subsequent FST domains characterized by the presence of clusters of positively charged basic amino acids that form ion pairs with spatially defined negatively charged sulfo- or carboxyl groups of the GAG chain. FST binds heparin at a highly basic 12-residue segment (residues 75-86) in the first FS domain. The complex localizes it to the cell surface and facilitates endocytosis of FST-bound ligands, thus acting as a neutralization mechanism of the ligand. There are no known receptors for FST, however the interaction with Activin A and Myostatin that are acting as ligands for FST is very well characterized.

Myostatin and Activin A are TGF-β family members that act to limit skeletal muscle mass. Myostatin and Activin A reduce the ability of the muscle to regenerate to acute and chronic injuries by suppressing the ability of the satellite cells to differentiate and fuse in thick muscle fibers. In addition, myostatin reduces proliferation of both young and aged bone marrow stem cells (BMSC) in a dose-dependent fashion while Activin A increases mineralization in both young and aged BMSCs. Activin A also directs the development of monocyte/macrophages, myeloid dendritic cells, and T cell subsets to promote type 2 and regulatory immune responses.

The serum data collected from patients suggest that aging is accompanied by changes in the expression of activin A and myostatin, as well as changes in the response of bone and muscle progenitor cells to these factors. Thus, inhibition of Myostatin and Activin A activity it is likely to be effective for increasing muscle mass and strength, while attenuate the immune deficiencies caused by senescence.

The disclosure further discloses an in-vitro cell culture system used to generate the CM. The system includes a partial differentiated cell population from human pluripotent or multipotent stem cells. The cells are initially exposed to a serum free media containing bFGF and Activin A. When reaching confluence, the bFGF and Activin A is removed from composition and the cultures allowed to differentiate in the presence of supraphysiological concentrations of signaling amino-acids Arginine and Leucine for 1-3 days. After differentiation, fresh media is added daily after collecting the supernatant. The supernatant is further used to produce the compositions described herein.

The analysis of the supernatant from 3 different cultures of partial differentiated pluripotent stem cells obtained as described above revealed 9.56+/−3.18 μg/L FST, and 2.86+/−0.42 mg/L of AHSG. The detected concentration of these proteins is much higher than the concentration in normal adult humans: FST 3.5+/−0.2 μg/L and AHSG: 0.58±0.12 mg/L.

An exemplary starting population is a pluripotent stem cell population originated from embryos (embryonic stem cells) or induced pluripotent stem cells. An exemplary species of cells as FST source is human, however given the highly conserved nature of the FST, a broad range of species can be used, including non-mammalian species (i.e. fish or insect). In certain embodiments, the species of AHSG and sulphated GAGs is human, given the potential immunogenic nature of the xenogenic molecules. Recombinant human proteins produced by various systems (bacterial, insect or mammalian) are also an exemplary alternative source of AHSG and follistatin for synthesis.

Table 1 provides exemplary sequences for inclusion in the AHSG molecules described and used herein.

TABLE 1
Fetuin A protein isoforms (excluding signal
peptide sequences; TGF-beta binding domain
underlined)

Fetuin A isoform 1 (NP_001341500.1;
SEQ ID NO: 1)
APHGPGLIYRQPNCDDPETEEAALVAIDYINQNLPWGYKHTLNQIDEVKV
WPQQPSGELFEIEIDTLETTCHVLDPTPVARCSVRQLKEHAVEGD
CDFQLLKLDGKFSVVYAKC
DSSPADSAEDVRKVCQDCPLLAPLNDTRVVHAAKAALAAFNAQNNGSNFQ
LEEISRAQLVPLPPSTYVEFTVSGTDCVAKEATEAAKCNLLAEKQYGFCK
ATLSEKLGGAEVAVTCMVFQTQPVSSQPQPEGANEAVPTPVVDPDAPPSP
PLGAPGLPPAGSPPDSHVL
(Chain A, SEQ ID NO: 5)
LAAPPGHQLHRAHYDLRHTEMGVVSLGSPSGEVSHPRKTR (linker,
SEQ ID NO: 6)
TVVQPSVGAAAGPVVPPCPGRIRHFKV (Chain B,
SEQ ID NO: 7)
Fetuin A isoform 2 (NP_001613.2; SEQ ID NO: 2)
APHGPGLIYRQPNCDDPETEEAALVAIDYINQNLPWGYKHTLNQIDEVKV
WPQQPSGELFEIEIDTLETTCHVLDPTPVARCSVRQLKEHAVEGD
CDFQLLKLDGKFSVVYAKC
DSSPDSAEDVRKVCQDCPLLAPLNDTRVVHAAKAALAAFNAQNNGSNFQL
EEISRAQLVPLPPSTYVEFTVSGTDCVAKEATEAAKCNLLAEKQYGFCKA
TLSEKLGGAEVAVTCMVFQTQPVSSQPQPEGANEAVPTPVVDPDAPPSPP
LGAPGLPPAGSPPDSHVL
(Chain A, SEQ ID NO: 8)
LAAPPGHQLHRAHYDLRHTEMGVVSLGSPSGEVSHPRKTR (linker,
SEQ ID NO: 6)
TVVQPSVGAAAGPVVPPCPGRIRHFKV (Chain B,
SEQ ID NO: 7)
Fetuin A isoform 3 (NP_001341501.1; SEQ ID NO: 3)
APHGPGLIYRQPNCDDPETEEAALVAIDYINQNLPWGYKHTLNQIDEVKV
WPQPSGELFEIEIDTLETTCHVLDPTPVARCSVRQLKEHAVEGD
CDFQLLKLDGKFSVVYAKC
DSSPDSAEDVRKVCQDCPLLAPLNDTRVVHAAKAALAAFNAQNNGSNFQL
EEISRAQLVPLPPSTYVEFTVSGTDCVAKEATEAAKCNLLAEKQYGFCKA
TLSEKLGGAEVAVTCMVFQTQPVSSQPQPEGANEAVPTPVVDPDAPPSPP
LGAPGLPPAGSPPDSHVL
(Chain A, SEQ ID NO: 9)
LAAPPGHQLHRAHYDLRHTFMGVVSLGSPSGEVSHPRKTR (linker,
SEQ ID NO: 6)
TVVQPSVGAAAGPVVPPCPGRIRHFKV (Chain B,
SEQ ID NO: 7)
Fetuin A isoform 4 (NP_001341502.1; SEQ ID NO: 4)
APHGPGLIYRQPNCDDPETEEAALVAIDYINQNLPWGYKHTLNQIDEVKV
WPQQPSGELFEIEIDTLETTCHVLDPTPVARCSVRQLKEHAVEGD
CDFQLLKLDGKFSVVYAKC
DSSPDSAEDVRKVCQDCPLLAPLNDTRVVHAAKAALAAFNAQNNGSNFQL
EEISRAQLVPLPPSTYVEFTVSGTDCVAKEATEAAKCNLLAEKPVSSQPQ
PEGANEAVPTPVVDPDAPPSPPLGAPGLPPAGSPPDSHVL
(Chain A, SEQ ID NO: 10)
LAAPPGHQLHRAHYDLRHTFMGVVSLGSPSGEVSHPRKTR (linker,
SEQ ID NO: 6)
TVVQPSVGAAAGPVVPPCPGRIRHFKV (Chain B,
SEQ ID NO: 7)
FETUA_HUMAN Alpha-2-HS-glycoprotein
(SEQ ID NO: 11)
MKSLVLLLCLAQLWGCHSAPHGPGLIYRQPNCDDPETEEAALVAIDYINQ
NLPWGYKHTLNQIDEVKVWPQQPSGELFEIEIDTLETTCHVLDPTPVARC
SVRQLKEHAVEGDCDFQLLKLDGKFSVVYAKC
DSSPDSAEDVRKVCQDCPLLAPLNDTRVVHAAKAALAAFNAQNNGSNFFC
QLEEISRAQLVPLPPSTYVEFTVSGTDCVAKEATEAAKCNLLAEKQYGKA
TLSEKLGGAEVAVTCTVFQTQPVTSQPQPEGANEAVPTPVVDPDAPPSPP
LGAPGLPPAGSPPDSHVLLAAPPGHQLHRAHYDLRHTFMGVVSLGSPSGE
VSHPRKTRTVVQPSVGAAAGPVVPPCPGRIRHFKV
TGF-beta binding domain (SEQ ID NO: 12)
CDFQLLKLDGKFSVVYAKC

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the FIGURES.

In many embodiments, a synthetic bioconjugate includes a sulphated GAG and one or more single chain, non-phosphorylated AHSG molecule or fragment of a single chain, non-phosphorylated AHSG molecule, wherein the one or more single chain, non-phosphorylated AHSG molecule or fragment of a single chain, non-phosphorylated AHSG molecule, are covalently conjugated to the sulphated GAG.

In some embodiments, the sulphated GAG is heparan sulfate (HS) heparin (HEP), chondroitin sulfate (CS), dermatan sulfate (DS), keratan sulfate (KS), or a derivative thereof.

In some embodiments, the sulphated GAG is a chemically modified GAG.

In some embodiments, the sulphated GAG is a chemically sulfated GAG.

In some embodiments, the one or more single chain, non-phosphorylated AHSG molecule or fragment of a single chain, non-phosphorylated AHSG molecule are covalently conjugated to the sulphated GAG at the N-terminus.

In some embodiments, the one or more single chain, non-phosphorylated AHSG molecule or fragment of a single chain, non-phosphorylated AHSG molecule are covalently conjugated to the sulphated GAG at the N-terminus via an amide bond.

In some embodiments, the one or more single chain, non-phosphorylated AHSG molecules or fragment of a single chain, non-phosphorylated AHSG molecule has at least 90% sequence identity to fetuin-A.

In some embodiments, the fragment of a single chain, non-phosphorylated AHSG molecule comprises a TGFβ-binding sequence of fetuin-A.

In some embodiments, the fragment of a single chain, non-phosphorylated AHSG molecule has at least 80% sequence identity, at least 90% sequence identity, at least 95% sequence identity, or at least 99% sequence identity, to the TGFβ-binding sequence of fetuin-A.

In some embodiments, the fragment of a single chain, non-phosphorylated AHSG molecule comprises an amino acid sequence selected from the group consisting of SEQ ID NO:1, SEQ ID NO:2, SEQ ID NO:3, SEQ ID NO:4, and SEQ ID NO:11.

In some embodiments, the fragment of a single chain, non-phosphorylated AHSG molecule comprises an amino acid sequence selected from the group consisting of SEQ ID NO:1, SEQ ID NO:2, SEQ ID NO:3, SEQ ID NO:4, and SEQ ID NO:11, or is a fragment of any one of SEQ ID NO:1-4 or SEQ ID NO:11 that comprises the fragment of SEQ ID NO:12.

In some embodiments, the fragment of a single chain, non-phosphorylated AHSG molecule comprises the amino acid sequence of SEQ ID NO:12 or an amino acid sequence having at least 85% sequence identity to SEQ ID NO:12.

In some embodiments, the fragment of a single chain, non-phosphorylated AHSG molecule has at least 80% sequence identity, at least 90% sequence identity, at least 95% sequence identity, or at least 99% sequence identity, to SEQ ID NO:12.

In some embodiments, the sulphated GAG comprises from about 1 to about 75 percent (%) functionalization, wherein the percent (%) functionalization is determined by a percent of disaccharide units on the sulphated GAG which are functionalized with a single chain, non-phosphorylated AHSG molecule or fragment of a single chain, non-phosphorylated AHSG molecule.

In some embodiments, the synthetic bioconjugate comprises from 1 to 20 single chain, non-phosphorylated AHSG molecules or fragment of a single chain, non-phosphorylated AHSG molecule.

In some embodiments, the synthetic bioconjugate comprises from 1 to 5 single chain, non-phosphorylated AHSG molecule or fragment of a single chain, non-phosphorylated AHSG molecule.

In some embodiments, the synthetic bioconjugate comprises one single chain, non-phosphorylated AHSG molecule or fragment of a single chain, non-phosphorylated AHSG molecule.

In some embodiments, the one or more single chain, non-phosphorylated AHSG molecule or fragment of a single chain, non-phosphorylated AHSG molecule are covalently conjugated to the sulphated GAG through a linker.

In some embodiments, the one or more single chain, non-phosphorylated AHSG molecule or fragment of a single chain, non-phosphorylated AHSG molecule are covalently conjugated to the backbone of the sulphated GAG through a linker.

In many embodiments, a synthetic bioconjugate includes a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of a single chain, non-phosphorylated AHSG molecule.

In many embodiments, a composition includes a synthetic bioconjugate of any preceding claim, wherein the average number of single chain, non-phosphorylated AHSG molecule or fragment of a single chain, non-phosphorylated AHSG molecule per sulphated GAG is from 1 to about 10.

In many embodiments, a composition includes a synthetic bioconjugate of any one of claims 1-20, wherein the average number of single chain, non-phosphorylated AHSG molecule or fragment of a single chain, non-phosphorylated AHSG molecule per sulphated GAG is from 1 to about 5.

In some embodiments, the composition further comprises follistatin.

In some embodiments, the follistatin is present in a 1- to 5-fold excess of the synthetic bioconjugate.

In many embodiments, a method for treating an age-related disease or disorder, comprising administering a patient in need thereof, an effective amount of the synthetic bioconjugate of any one of claims 1-20, or the composition of claim 21 or 22.

In some embodiments, a method for treating a disease affecting the osteo-muscular system, comprising administering a patient in need thereof, an effective amount of the synthetic bioconjugate of any one of claims 1-20, or the composition of claim 21 or 22.

In many embodiments, a method for treating an immune dysregulation, comprising administering a patient in need thereof, an effective amount of the synthetic bioconjugate of any one of claims 1-20, or the composition of claim 30 or 31.

In many embodiments, a method of any one of claims 21-23, wherein the synthetic bioconjugate of any one of claims 1-16, or the composition of claim 21 or 22 is administered intramuscularly, intraarticularly, or intravenously.

In some embodiments, the synthetic bioconjugate of any one of claims 1-16, or the composition of claim 17 or 18 is administered in an amount ranging from about 0.01 to about 2.5 mg/kg per body weight.

In many embodiments, a composition includes a sulphated GAG backbone functionalized with at least one AHSG molecule, wherein the sulphated GAG backbone is heparan sulfate (HS), heparin (HEP), chondroitin sulfate (CS), dermatan sulfate (DS), or keratan sulfate (KS), and wherein the at least one AHSG molecule is single chain and non-phosphorylated.

In some embodiments, the sulphated GAG backbone binds a peptide with a heparin binding domain, and wherein the peptide is follistatin (FST). In some embodiments, the follistatin is a natural molecule collected from a cell culture, or a synthetic peptide with similar functionality of binding Activin A and Myostatin. In some embodiments, the sulphated GAG backbone, the at least one AHSG molecule, and FST are obtained in the same in vitro cell culture. In some embodiments, the in vitro cell culture is derived from human stem cells, and wherein the human stem cells are embryonic or induced pluripotent stem cells.

In many embodiments, a method to produce a molecular structure includes the steps of: coupling carboxyl terminations of a GAG molecule with an N-terminus of at least one AHSG molecule using a crosslinker to produce a crosslinked structure; removing an excess amount of the crosslinker by dialysis; coupling the crosslinked structure and a growth factor; and diluting the coupled crosslinked structure and growth factor in a physiological buffer.

In some embodiments, the growth factor comprises follistatin.

In many embodiments, a method to produce a molecular structure includes the steps of: obtaining a cell culture supernatant comprising at least one GAG molecules and at least one AHSG molecule; removing molecules below 10 kDa by dialysis to create a dialyzed supernatant comprising the at least one GAG molecules and the at least one AHSG molecule; mixing a carbodiimide crosslinker with the dialyzed supernatant, wherein carboxyl terminations of the at least one GAG molecule are coupled with N-termini of the at least one AHSG molecule to produce a crosslinked structure; and removing an excess amount of the carbodiimide crosslinker by dialysis.

In some embodiments, the method further includes the steps of adding additional peptides to the dialyzed supernatant before mixing the carbodiimide crosslinker with the dialyzed supernatant.

In many embodiments, an injectable medical preparation includes a synthetic bioconjugate comprising a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of a single chain, non-phosphorylated AHSG molecule; and a pharmaceutically acceptable carrier.

In some embodiments, the preparation also includes amino-acids.

In some embodiments, the preparation also includes small peptides.

In some embodiments, the preparation is lyophilized.

In some embodiments, the preparation is sterilized by gamma irradiation.

In some embodiments, the preparation is packaged in a single use medical device.

In many embodiments, a method of treating a medical condition includes the steps of: administering a composition comprising a synthetic bioconjugate comprising a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of a single chain, non-phosphorylated AHSG molecule; and a pharmaceutically acceptable carrier.

In some embodiments, the composition is administered by injection.

In some embodiments, the medical condition is a disease affecting the osteo-muscular system.

In some embodiments, the medical condition is an immune dysregulation.

In some embodiments, the medical condition is age related.

### CLAUSES

Exemplary embodiments are set out in the following numbered clauses.
Clause 1. A synthetic bioconjugate comprising a sulphated GAG and one or more single chain, non-phosphorylated AHSG molecule or fragment of a single chain, non-phosphorylated AHSG molecule, wherein the one or more single chain, non-phosphorylated AHSG molecule or fragment of a single chain, non-phosphorylated AHSG molecule, are covalently conjugated to the sulphated GAG.
Clause 2. The synthetic bioconjugate of clause 1, wherein the sulphated GAG is heparan sulfate (HS) heparin (HEP), chondroitin sulfate (CS), dermatan sulfate (DS), keratan sulfate (KS), or a derivative thereof.
Clause 3. The synthetic bioconjugate of clause 2 or 3, wherein the sulphated GAG is a chemically modified GAG.
Clause 4. The synthetic bioconjugate of any preceding clause, wherein the sulphated GAG is a chemically sulfated GAG.
Clause 5. The synthetic bioconjugate of any preceding clause, wherein the one or more single chain, non-phosphorylated AHSG molecule or fragment of a single chain, non-phosphorylated AHSG molecule are covalently conjugated to the sulphated GAG at the N-terminus.
Clause 6. The synthetic bioconjugate of any preceding clause, wherein the one or more single chain, non-phosphorylated AHSG molecule or fragment of a single chain, non-phosphorylated AHSG molecule are covalently conjugated to the sulphated GAG at the N-terminus via an amide bond.
Clause 7. The synthetic bioconjugate of any preceding clause, wherein the one or more single chain, non-phosphorylated AHSG molecules or fragment of a single chain, non-phosphorylated AHSG molecule has at least 90% sequence identity to fetuin-A.
Clause 8. The synthetic bioconjugate of any one of clauses 1-7, wherein the fragment of a single chain, non-phosphorylated AHSG molecule comprises a TGFβ-binding sequence of fetuin-A.
Clause 9. The synthetic bioconjugate of clause 8, wherein the fragment of a single chain, non-phosphorylated AHSG molecule has at least 80% sequence identity, at least 90% sequence identity, at least 95% sequence identity, or at least 99% sequence identity, to the TGFβ-binding sequence of fetuin-A.
Clause 10. The synthetic bioconjugate of any one of clauses 1-9, wherein the fragment of a single chain, non-phosphorylated AHSG molecule comprises an amino acid sequence selected from the group consisting of SEQ ID NO:1, SEQ ID NO:2, SEQ ID NO:3, SEQ ID NO:4, and SEQ ID NO:11.
Clause 11. The synthetic bioconjugate of any one of clauses 1-9, wherein the fragment of a single chain, non-phosphorylated AHSG molecule comprises an amino acid sequence selected from the group consisting of SEQ ID NO:1, SEQ ID NO:2, SEQ ID NO:3, SEQ ID NO:4, and SEQ ID NO:11, or is a fragment of any one of SEQ ID NO:1-4 or SEQ ID NO:11 that comprises the fragment of SEQ ID NO:12.
Clause 12. The synthetic bioconjugate of any one of clauses 1-9, wherein the fragment of a single chain, non-phosphorylated AHSG molecule comprises the amino acid sequence of SEQ ID NO:12 or an amino acid sequence having at least 85% sequence identity to SEQ ID NO:12.
Clause 13. The synthetic bioconjugate of any one of clauses 1-9, wherein the fragment of a single chain, non-phosphorylated AHSG molecule has at least 80% sequence identity, at least 90% sequence identity, at least 95% sequence identity, or at least 99% sequence identity, to SEQ ID NO:12.
Clause 14. The synthetic bioconjugate of any preceding clause, wherein the sulphated GAG comprises from about 1 to about 75 percent (%) functionalization, wherein the percent (%) functionalization is determined by a percent of disaccharide units on the sulphated GAG which are functionalized with a single chain, non-phosphorylated AHSG molecule or fragment of a single chain, non-phosphorylated AHSG molecule.
Clause 15. The synthetic bioconjugate of any preceding clause, wherein the synthetic bioconjugate comprises from 1 to 20 single chain, non-phosphorylated AHSG molecules or fragment of a single chain, non-phosphorylated AHSG molecule.
Clause 16. The synthetic bioconjugate of any preceding clause, wherein the synthetic bioconjugate comprises from 1 to 5 single chain, non-phosphorylated AHSG molecule or fragment of a single chain, non-phosphorylated AHSG molecule.
Clause 17. The synthetic bioconjugate of any preceding clause, wherein the synthetic bioconjugate comprises one single chain, non-phosphorylated AHSG molecule or fragment of a single chain, non-phosphorylated AHSG molecule.
Clause 18. The synthetic bioconjugate of any preceding clause, wherein the one or more single chain, non-phosphorylated AHSG molecule or fragment of a single chain, non-phosphorylated AHSG molecule are covalently conjugated to the sulphated GAG through a linker.
Clause 19. The synthetic bioconjugate of any preceding clause, wherein the one or more single chain, non-phosphorylated AHSG molecule or fragment of a single chain, non-phosphorylated AHSG molecule are covalently conjugated to the backbone of the sulphated GAG through a linker.
Clause 20. A synthetic bioconjugate comprising a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of a single chain, non-phosphorylated AHSG molecule.
Clause 21. A composition comprising a synthetic bioconjugate of any preceding clause, wherein the average number of single chain, non-phosphorylated AHSG molecule or fragment of a single chain, non-phosphorylated AHSG molecule per sulphated GAG is from 1 to about 10.
Clause 22. A composition comprising a synthetic bioconjugate of any one of clauses 1-20, wherein the average number of single chain, non-phosphorylated AHSG molecule or fragment of a single chain, non-phosphorylated AHSG molecule per sulphated GAG is from 1 to about 5.
Clause 23. A composition comprising the synthetic bioconjugate of any one of clauses 1-20, or the composition of clause 21 or 22, wherein the composition further comprises follistatin.
Clause 24. The composition of clause 23, wherein the follistatin is present in a 1- to 5-fold excess of the synthetic bioconjugate.
Clause 25. A method for treating an age-related disease or disorder, comprising administering a patient in need thereof, an effective amount of the synthetic bioconjugate of any one of clauses 1-20, or the composition of clause 21 or 22.
Clause 26. A method for treating a disease affecting the osteo-muscular system, comprising administering a patient in need thereof, an effective amount of the synthetic bioconjugate of any one of clauses 1-20, or the composition of clause 21 or 22.
Clause 27. A method for treating an immune dysregulation, comprising administering a patient in need thereof, an effective amount of the synthetic bioconjugate of any one of clauses 1-20, or the composition of clause 30 or 31.
Clause 28. The method of any one of clauses 21-23, wherein the synthetic bioconjugate of any one of clauses 1-16, or the composition of clause 21 or 22 is administered intramuscularly, intraarticularly, or intravenously.
Clause 29. The method of clause 24, wherein the synthetic bioconjugate of any one of clauses 1-16, or the composition of clause 17 or 18 is administered in an amount ranging from about 0.01 to about 2.5 mg/kg per body weight.
Clause 30. A composition comprising a sulphated GAG backbone functionalized with at least one AHSG molecule, wherein the sulphated GAG backbone is heparan sulfate (HS), heparin (HEP), chondroitin sulfate (CS), dermatan sulfate (DS), or keratan sulfate (KS), and wherein the at least one AHSG molecule is single chain and non-phosphorylated.
Clause 31. The composition of clause 30, wherein the sulphated GAG backbone binds a peptide with a heparin binding domain, and wherein the peptide is follistatin (FST).
Clause 32. The composition of clause 31, wherein the follistatin is a natural molecule collected from a cell culture, or a synthetic peptide with similar functionality of binding Activin A and Myostatin.
Clause 33. The composition of clause 31, wherein the sulphated GAG backbone, the at least one AHSG molecule, and FST are obtained in the same in vitro cell culture.
Clause 34. The composition of clause 33, wherein the in vitro cell culture is derived from human stem cells, and wherein the human stem cells are embryonic or induced pluripotent stem cells.
Clause 35. A method to produce a molecular structure, comprising the steps of:
   coupling carboxyl terminations of a GAG molecule with an N-terminus of at least one AHSG molecule using a crosslinker to produce a crosslinked structure;
   removing an excess amount of the crosslinker by dialysis;
   coupling the crosslinked structure and a growth factor; and
   diluting the coupled crosslinked structure and growth factor in a physiological buffer.
Clause 36. The method of clause 35, wherein the growth factor comprises follistatin.
Clause 37. A method to produce a molecular structure, comprising the steps of:
   obtaining a cell culture supernatant comprising at least one GAG molecules and at least one AHSG molecule;
   removing molecules below 10 kDa by dialysis to create a dialyzed supernatant comprising the at least one GAG molecules and the at least one AHSG molecule;
   mixing a carbodiimide crosslinker with the dialyzed supernatant, wherein carboxyl terminations of the at least one GAG molecule are coupled with N-termini of the at least one AHSG molecule to produce a crosslinked structure; and removing an excess amount of the carbodiimide crosslinker by dialysis.
Clause 38. The method of clause 37, further comprising the step of adding additional peptides to the dialyzed supernatant before mixing the carbodiimide crosslinker with the dialyzed supernatant.
Clause 39. An injectable medical preparation, comprising:
   a synthetic bioconjugate comprising a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of a single chain, non-phosphorylated AHSG molecule; and
   a pharmaceutically acceptable carrier.
Clause 40. The injectable medical preparation of clause 39, further comprising amino-acids.
Clause 41. The injectable medical preparation of clause 39, further comprising small peptides.
Clause 42. The injectable medical preparation of clause 39, wherein the preparation is lyophilized.
Clause 43. The injectable medical preparation of clause 39, wherein the preparation is sterilized by gamma irradiation.
Clause 44. The injectable medical preparation of clause 39, wherein the preparation is packaged in a single use medical device.
Clause 45. A method of treating a medical condition, comprising the steps of:
   administering a composition comprising a synthetic bioconjugate comprising a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of a single chain, non-phosphorylated AHSG molecule; and a pharmaceutically acceptable carrier.
Clause 46. The method of clause 45, wherein the composition is administered by injection.
Clause 47. The method of clause 45, wherein the medical condition is a disease affecting the osteo-muscular system.
Clause 48. The method of clause 45, wherein the medical condition is an immune dysregulation.
Clause 49. The method of clause 45, wherein the medical condition is age related.

### REFERENCES

Sakamoto Y, Shintani Y, Harada K, Abe M, Shitsukawa K, Saito S. Determination of free follistatin levels in sera of normal subjects and patients with various diseases. Eur J Endocrinol. 1996 Sep.; 135(3):345-51.
Wang, Z., Wang, Z., Lu, W. et al. Novel biomaterial strategies for controlled growth factor delivery for biomedical applications. NPG Asia Mater 9, e435 (2017) doi:10.1038/am.2017.171
Bowser Ml, Herberg S, Arounleut P, Shi X, Fulzele S, Hill W D, Isales CM, Hamrick MW. Effects of the activin A-myostatin-follistatin system on aging bone and muscle progenitor cells. Exp Gerontol. 2013 Feb.; 48(2):290-7. doi: 10.1016/j.exger.2012.11.004. Epub 2012 Nov. 21.

I claim:

1. A synthetic bioconjugate comprising a sulphated GAG and one or more single chain, non-phosphorylated AHSG molecule or fragment of a single chain, non-phosphorylated AHSG molecule, wherein the one or more single chain, non-phosphorylated AHSG molecule or fragment of the single chain, non-phosphorylated AHSG molecule, are covalently conjugated to the sulphated GAG.

2. The synthetic bioconjugate of claim 1, wherein the sulphated GAG is heparan sulfate (HS), heparin (HEP), chondroitin sulfate (CS), dermatan sulfate (DS), keratan sulfate (KS), or a derivative thereof.

3. The synthetic bioconjugate of claim 1, wherein the sulphated GAG is a chemically modified GAG.

4. The synthetic bioconjugate of claim 1, wherein the sulphated GAG is a chemically sulfated GAG.

5. The synthetic bioconjugate of claim 1, wherein the one or more single chain, non-phosphorylated AHSG molecule or fragment of the single chain, non-phosphorylated AHSG molecule are covalently conjugated to the sulphated GAG at the N-terminus.

6. The synthetic bioconjugate of claim 1, wherein the one or more single chain, non-phosphorylated AHSG molecule or fragment of the single chain, non-phosphorylated AHSG molecule are covalently conjugated to the sulphated GAG at the N-terminus via an amide bond.

7. The synthetic bioconjugate of claim 1, wherein the one or more single chain, non-phosphorylated AHSG molecules or fragment of the single chain, non-phosphorylated AHSG molecule has at least 90% sequence identity to fetuin-A.

8. The synthetic bioconjugate of claim 1, wherein the fragment of the single chain, non-phosphorylated AHSG molecule comprises a TGFβ-binding sequence of fetuin-A.

9. The synthetic bioconjugate of claim 8, wherein the fragment of the single chain, non-phosphorylated AHSG molecule has at least 80% sequence identity, at least 90% sequence identity, at least 95% sequence identity, or at least 99% sequence identity, to the TGFβ-binding sequence of fetuin-A.

10. The synthetic bioconjugate of claim 1, wherein the fragment of the single chain, non-phosphorylated AHSG molecule comprises an amino acid sequence selected from the group consisting of SEQ ID NO:1, SEQ ID NO:2, SEQ ID NO:3, SEQ ID NO:4, and SEQ ID NO:11.

11. The synthetic bioconjugate of claim 1, wherein the fragment of the single chain, non-phosphorylated AHSG molecule comprises an amino acid sequence selected from the group consisting of SEQ ID NO:1, SEQ ID NO:2, SEQ ID NO:3, SEQ ID NO:4, and SEQ ID NO:11, or is a fragment of any one of SEQ ID NO:1-4 or SEQ ID NO:11 that comprises the fragment of SEQ ID NO:12.

12. The synthetic bioconjugate of claim 1, wherein the fragment of the single chain, non-phosphorylated AHSG molecule comprises the amino acid sequence of SEQ ID NO:12 or an amino acid sequence having at least 85% sequence identity to SEQ ID NO:12.

13. The synthetic bioconjugate of claim 1, wherein the fragment of the single chain, non-phosphorylated AHSG molecule has at least 80% sequence identity, at least 90% sequence identity, at least 95% sequence identity, or at least 99% sequence identity, to SEQ ID NO:12.

14. The synthetic bioconjugate of claim 1, wherein the sulphated GAG comprises from about 1 to about 75 percent (%) functionalization, wherein the percent (%) functionalization is determined by a percent of disaccharide units on the sulphated GAG which are functionalized with a single chain, non-phosphorylated AHSG molecule or fragment of the single chain, non-phosphorylated AHSG molecule.

15. The synthetic bioconjugate of claim 1, wherein the synthetic bioconjugate comprises from 1 to 20 single chain, non-phosphorylated AHSG molecules or fragment of the single chain, non-phosphorylated AHSG molecule.

16. The synthetic bioconjugate of claim 1, wherein the synthetic bioconjugate comprises from 1 to 5 single chain, non-phosphorylated AHSG molecule or fragment of the single chain, non-phosphorylated AHSG molecule.

17. The synthetic bioconjugate of claim 1, wherein the synthetic bioconjugate comprises one single chain, non-phosphorylated AHSG molecule or fragment of the single chain, non-phosphorylated AHSG molecule.

18. The synthetic bioconjugate of claim 1, wherein the one or more single chain, non-phosphorylated AHSG molecule or fragment of the single chain, non-phosphorylated AHSG molecule are covalently conjugated to the sulphated GAG through a linker.

19. The synthetic bioconjugate of claim 1, wherein the one or more single chain, non-phosphorylated AHSG molecule or fragment of the single chain, non-phosphorylated AHSG molecule are covalently conjugated to the backbone of the sulphated GAG through a linker.

20. A synthetic bioconjugate comprising at least one single chain, non-phosphorylated AHSG molecule or fragments of at least one single chain, non-phosphorylated AHSG molecule, and one or more sulphated GAGs, wherein the one or more sulphated GAGs are covalently conjugated to the single chain, non-phosphorylated AHSG molecule or fragment of the single chain, non-phosphorylated AHSG molecule.

21. The synthetic bioconjugate of claim 20, wherein an average number of the at least one single chain, non-phosphorylated AHSG molecule or the fragments of the at least one single chain, non-phosphorylated AHSG molecule per sulphated GAG is from 1 to about 10.

22. The synthetic bioconjugate of claim 20, wherein an average number of the at least one single chain, non-phosphorylated AHSG molecule or the fragments of the at least one single chain, non-phosphorylated AHSG molecule per sulphated GAG is from 1 to about 5.

23. The synthetic bioconjugate of claim 20, wherein the bioconjugate further comprises follistatin.

24. The synthetic bioconjugate of claim 23, wherein the follistatin is present in a 1- to 5-fold excess of the sulphated GAG.

25. A method for treating a medical condition in a patient, comprising the step of:
administering a patient in need thereof, an effective amount of a synthetic bioconjugate comprising at least one single chain, non-phosphorylated AHSG molecule or fragments of at least one single chain, non-phosphorylated AHSG molecule, and one or more sulphated GAGs, wherein the one or more sulphated GAGs are covalently conjugated to the single chain, non-phosphorylated AHSG molecule or fragment of the single chain, non-phosphorylated AHSG molecule.

26. The method of claim 25, wherein the medical condition is an age-related disease or disorder.

27. The method of claim 25, wherein the medical condition is a disease affecting the osteo-muscular system.

28. The method of claim 25, wherein the medical condition is an immune dysregulation.

29. The method of claim 25, wherein the synthetic bioconjugate is administered intramuscularly, intraarticularly, or intravenously.

30. The method of claim 25, wherein the synthetic bioconjugate is administered in an amount ranging from about 0.01 to about 2.5 mg/kg per body weight.

31. A composition comprising a sulphated GAG backbone functionalized with at least one AHSG molecule, wherein the sulphated GAG backbone is heparan sulfate (HS), heparin (HEP), chondroitin sulfate (CS), dermatan sulfate (DS), or keratan sulfate (KS), and wherein the at least one AHSG molecule is in a single chain and non-phosphorylated form.

32. The composition of claim 31, wherein the composition further comprises a peptide with a heparin binding domain, wherein the sulphated GAG backbone binds the peptide with a heparin binding domain, and wherein the peptide with the heparin binding domain is follistatin (FST).

33. The composition of claim 32, wherein the follistatin is a natural molecule collected from a cell culture, or a synthetic peptide with similar functionality of binding Activin A and Myostatin.

34. The composition of claim 32, wherein the sulphated GAG backbone, the at least one AHSG molecule, and FST are obtained from a single in vitro cell culture.

35. The composition of claim 34, wherein the single in vitro cell culture is derived from human stem cells, and wherein the human stem cells are embryonic or induced pluripotent stem cells.

36. A method to produce a molecular structure, comprising the steps of:
coupling carboxyl terminations of a GAG molecule with an N-terminus of at least one AHSG molecule using a crosslinker to produce a crosslinked structure;
removing an excess amount of the crosslinker by dialysis;
coupling the crosslinked structure and a growth factor; and
diluting the coupled crosslinked structure and growth factor in a physiological buffer.

37. The method of claim 36, wherein the growth factor comprises follistatin.

38. A method to produce a molecular structure, comprising the steps of:
obtaining a cell culture supernatant comprising at least one GAG molecules and at least one AHSG molecule;
removing molecules below 10 kDa by dialysis to create a dialyzed supernatant comprising the at least one GAG molecules and the at least one AHSG molecule;
mixing a carbodiimide crosslinker with the dialyzed supernatant, wherein carboxyl terminations of the at least one GAG molecule are coupled with N-termini of the at least one AHSG molecule to produce a crosslinked structure; and
removing an excess amount of the carbodiimide crosslinker by dialysis.

39. The method of claim 38, further comprising the step of adding additional peptides to the dialyzed supernatant before mixing the carbodiimide crosslinker with the dialyzed supernatant.

40. An injectable medical preparation, comprising:
a synthetic bioconjugate comprising a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of the single chain, non-phosphorylated AHSG molecule; and
a pharmaceutically acceptable carrier.

41. The injectable medical preparation of claim 40, further comprising amino-acids.

42. The injectable medical preparation of claim 40, further comprising small peptides.

43. The injectable medical preparation of claim 40, wherein the preparation is lyophilized.

44. The injectable medical preparation of claim 40, wherein the preparation is sterilized by gamma irradiation.

45. The injectable medical preparation of claim 40, wherein the preparation is packaged in a single use medical device.

46. A method of treating a medical condition, comprising the steps of:
administering a composition comprising a synthetic bioconjugate comprising a single chain, non-phosphorylated AHSG molecule or fragment of a single chain, non-phosphorylated AHSG molecule, and one or more sulphated GAGs, wherein the one or more sulphated GAGs are covalently conjugated to the single chain, non-phosphorylated AHSG molecule or fragment of the single chain, non-phosphorylated AHSG molecule; and a pharmaceutically acceptable carrier.

47. The method of claim 46, wherein the composition is administered by injection.

48. The method of claim 46, wherein the medical condition is a disease affecting the osteo-muscular system.

49. The method of claim 46, wherein the medical condition is an immune dysregulation.

50. The method of claim 46, wherein the medical condition is age related.
